(19) United States
(12) Plant Patent Application Publication
(10) Pub. No.: US 2005/0183175 P1
(43) Pub. Date: Aug. 18, 2005

(54) Ground cover rose plant named 'NOA97400A'

(50) ROSA HYBRIDA; ROSE

(76) Inventor: Reinhard Noack, Gutersloh (DE)

Correspondence Address: BURNS DOANE SWECKER & MATHIS L L P, POST OFFICE BOX 1404, ALEXANDRIA, VA, 22313-1404, US

(21) Appl. No.: 11/043,094

(22) Filed: Jan. 27, 2005

(30) Foreign Application Priority Data
Feb. 14, 2004 (DE) ROS2408

(51) Int. Cl. A01H 5/00

(52) U.S. Cl. PLT/105

(57) ABSTRACT

A new and distinct variety for Ground Cover Rose plant is provided which forms on a substantially continuous basis sprays of attractive bright amber and cream-orange blossoms with the reproductive organs commonly being visible at the center. The blossoms open on a reliable basis and retain their attractiveness upon aging. The new variety initially exhibits a dense and upright growth habit that changes to a spreading growth habit during the second year. The foliage is dark green and glossy and contrasts well with the blossom coloration. The surfaces of leaves commonly are somewhat curled and partially closed. The blossom petals commonly detach cleanly upon full maturity. The plant propagates well from cuttings and by budding. Excellent resistance to Blackspot, Powdery Mildew, and Rust are displayed, and the plant is particularly well suited for growing as an attractive ornamental ground cover in the landscape.

### BOTANICAL/COMMERCIAL CLASSIFICATION

Rosa hybrida/Ground Cover Rose Plant

### VARIETAL DENOMINATION

cv. ‘NOA97400A’

### SUMMARY OF THE INVENTION

Ground Cover rose plants are known and are being used to advantage in an increasing number of landscape plans and home gardens. However, there remains a need for additional varieties of Ground Cover roses with the demand being the greatest for those having highly attractive blossoms in combination with good disease resistance.

The new variety of Rosa hybrida Ground Cover rose plant of the present invention was created at Gütersloh, Germany, by artificial pollination wherein two parents were crossed which previously had been studied in the hope that they would contribute the desired characteristics. The female parent (i.e., the seed parent) was the ‘Noatraum’ variety (U.S. Plant Pat. No. 7,282). The ‘Noatraum’ variety sometimes is identified as FLOWER CARPET PINK and is marketed in Europe under the HEIDETRAUM trademark. The male parent (i.e., the pollen parent) was an unnamed and unreleased seedling maintained by Noack Rosen at Gütersloh, Germany (non-patented in the United States). The parentage of the new variety can be summarized as follows:
‘Noatraum’×Unnamed Seedling.

The seeds resulting from the above pollination were sown and small plants were obtained which physically and biologically differ from each other. Selective study resulted in the identification of a single plant to the new variety.

It is found that the new variety of Ground Cover rose plant possesses the following combination of characteristics:

(a) forms in sprays attractive long-lasting bright amber and cream-orange blossoms with the reproductive organs commonly being visible at the center,

(b) initially exhibits a dense and upright growth habit that changes to a spreading growth habit during the second year,

(c) forms attractive glossy dark green foliage that contrasts well with the blossoms wherein the surfaces of the leaflets commonly are somewhat curled and partially closed,

(d) exhibits excellent disease resistance with respect to Blackspot, Powdery Mildew, and Rust, and

(e) is particularly well suited for growing as attractive ornamentation in the landscape.

The new variety meets the needs of the horticultural industry and can be grown to advantage as an attractive ornamental ground cover in parks, gardens, public areas, and residential landscapes.

The new variety of the present invention beginning in 2000 has been asexually reproduced by the rooting of cuttings, and by budding. Such asexual reproduction as performed at Gütersloh, Germany, has demonstrated that the characteristics of the new variety are firmly fixed and stable and are strictly transmissible from one generation to another. The new variety asexually reproduces in a true to type manner from one generation to another.

The new variety of the present invention has been named ‘NOA97400A’.

### BRIEF DESCRIPTION OF THE PHOTOGRAPHS

The accompanying photographs show as nearly true as it is reasonably possible to make the same, in color illustrations of this character, typical specimens of the new variety while growing outdoors. The illustrated rose plants were photographed during July, 2004, while growing in a field at Gütersloh, Germany. Such plants had been budded approximately one and one-half years earlier.

FIG. 1 illustrates a close view of the attractive amber and cream-orange blossoms of the new variety wherein the reproductive organs are visible at the center.

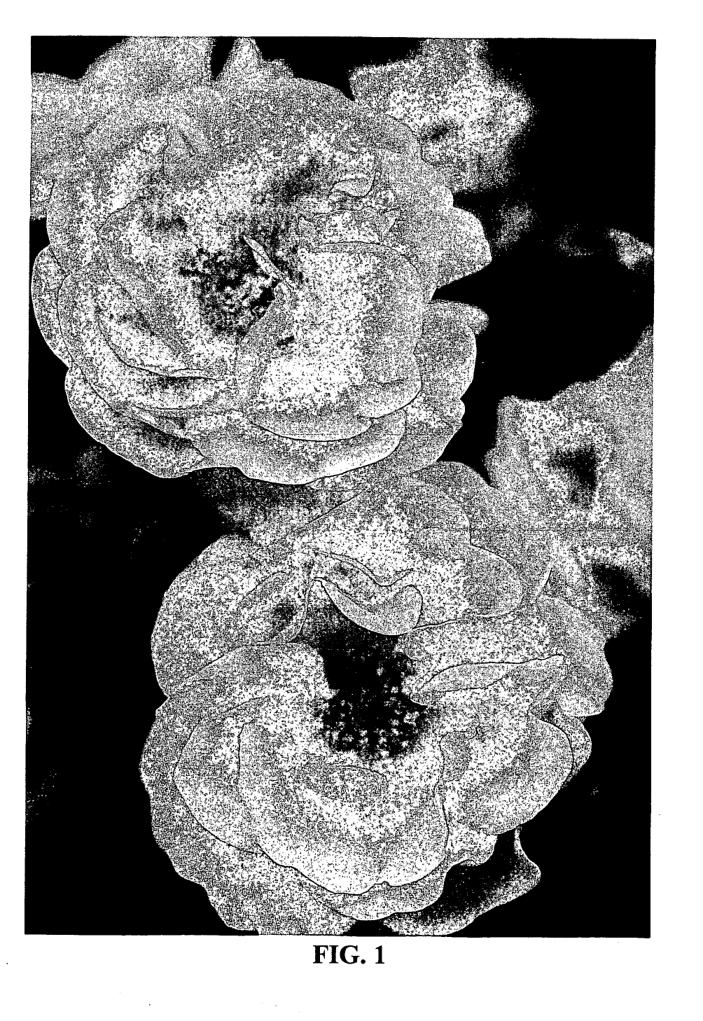

FIG. 2 illustrates at the right the upper and under surfaces of typical leaves, buds and glossy foliage, and at the left buds and flowers in various stages of opening as well as additional foliage. The configuration of the somewhat curled and partially closed leaflets is illustrated. Dimensions in centimeters are included at the center.

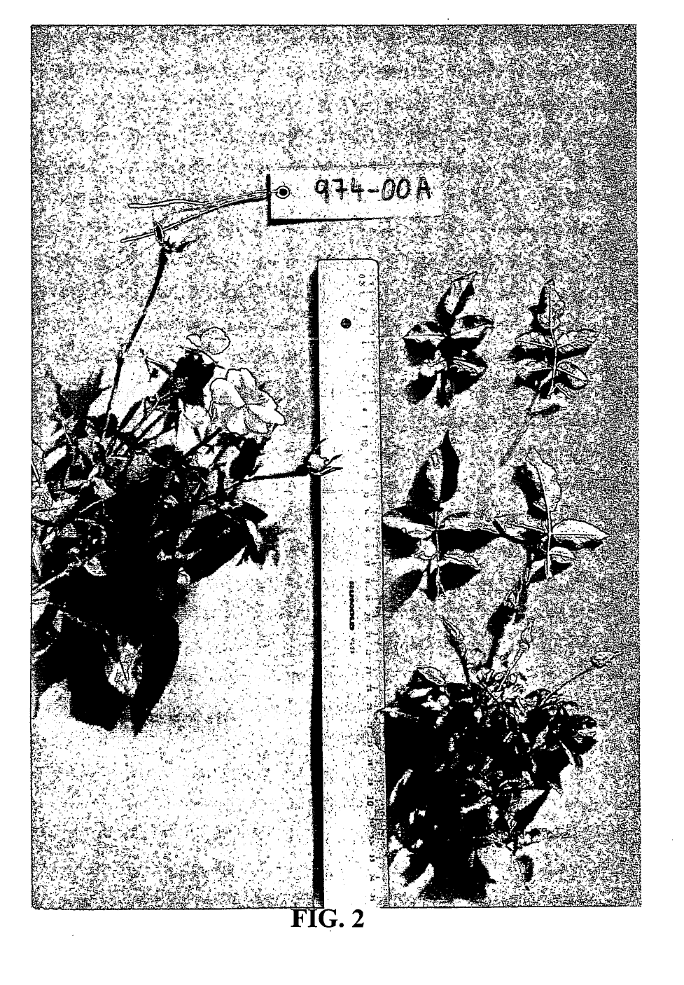

### DETAILED DESCRIPTION

The chart used in the identification of the colors is that of The Royal Horticultural Society (R.H.S. Colour Chart) of London, England. Color terminology in common terms sometimes is included as an aid to the reader. Such color terminology is to be accorded its customary dictionary significance. The description is based on the observation of typical specimens of the new variety while growing outdoors during July at Gütersloh, Germany.
Class: Ground Cover.
Plant:
   Form.—vigorous, young plants exhibit a dense and upright growth habit, and with further maturity spread to form a compact bush that serves well as ground cover with dense foliage.
   Height.—approximately 70 cm on average.
   Width.—approximately 70 cm on average.
Branches:
   Color.—young stems: medium green with a smooth surface. — adult wood: darker green than the young stems and with a smooth surface.
   Thorns.—size: medium, approximately 5 mm in length on average, and slightly curved downward on the upper surface, and concave on the under surface. — position: irregular. — color: initially light red-brown when immature and changing to dark brown at maturity. — quantity: typical for a Ground Cover rose plant.
Leaves:
   Stipules.—green in coloration.
   Leaflets.—number: commonly 5, and sometimes 7. — configuration: generally elliptical with a narrowly pointed apex, and commonly curled upwards at the margin thereby giving the leaves partially closed appearance. — quantity: very abundant. — size: medium. — serration: slightly serrate. — color: (young foliage): upper surface: medium green. under surface: lighter green than the upper surface. — color: (adult foliage): upper surface: dark green. under surface: lighter green than the upper surface. — general appearance: dense, dark green foliage that is glossy on the upper surface and matte on the under surface with the curled nature of the leaves being readily apparent. — texture: leathery. — stipules: light green in coloration.
Inflorescence:
   Number of flowers.—in clusters of flowers as a large spray.
   Peduncle.—soft green in coloration.
   Sepals.—shape: commonly extend well beyond the petals of the bud, pointed, and approximately 2 cm in length on average.
   Buds.—color when opening: Yellow-Orange Group 17A to 17D on the upper surface and Yellow-Orange Group 16C and 16D on the under surface.
   Flower.—form: somewhat flattened. — appearance: smooth on both petal surfaces. — diameter: approximately 5 cm on average. — color (when opening begins): upper surface: predominantly Yellow-Orange Group 17A. — color (when blooming): upper surface: Yellow-Orange Group 17A. The bright yellow coloration of pollen and the reproductive organs commonly is visible at the center. under surface: predominantly Yellow-Orange Group 16C and 16D. — color (at the end of opening): upper surface: bright amber and cream-orange coloration that is predominantly Yellow-Orange Group 17A. — fragrance: none. — lasting quality: very good. — petal form: broad, fan-shaped, curved with a somewhat indented central area and margin areas that are slightly curved towards the center. — petal number: commonly approximately 15 on average. — petal arrangement: generally arranged in a regular pattern with overlapping edges. — petal drop: good with the petals detaching cleanly upon fully maturity. — stamens: regularly arranged around the styles. — filaments: yellow in coloration. — pollen: yellow in coloration. — stigmas: yellow in coloration. — styles: thin and regularly arranged at the center. — hips: generally pear-shaped, approximately 2 cm in length, and initially green in coloration changing to red-orange with maturity.
Development:
   Vegetation.—strong, compact, and vigorous.
   Blooming.—substantially continuous throughout the season in large sprays.
   Aptitude to bear fruit.—slight.
   Winter hardiness.—good.
   Resistance to diseases.—excellent with respect to Blackspot, Powdery Mildew, and Rust.

1. A new and distinct Ground Cover Rose plant characterized by the following combination of characteristics:
(a) forms in sprays attractive long-lasting bright amber and cream-orange blossoms with the reproductive organs commonly being visible at the center,
(b) initially exhibits a dense and upright growth habit that changes to a spreading growth habit during the second year,
(c) forms attractive glossy dark green foliage that contrasts well with the blossoms wherein the surfaces of the leaflets commonly are somewhat curled and partially closed,
(d) exhibits excellent disease resistance with respect to Blackspot, Powdery Mildew, and Rust, and
(e) is particularly well suited for growing as attractive ornamentation in the landscape;
substantially as illustrated and described.
